(19) United States
(12) Patent Application Publication
(10) Pub. No.: US 2020/0109200 A1
(43) Pub. Date: Apr. 9, 2020

(54) METHODS AND SYSTEMS FOR DETERMINING SYNAPSE FORMATION

(71) Applicant: GENENTECH, INC., South San Francisco, CA (US)

(72) Inventors: Lynn Kamen, South San Francisco, CA (US); Brendan Bender, South San Francisco, CA (US); Shang-Chiung Chen, South San Francisco, CA (US)

(73) Assignee: GENENTECH, INC., South San Francisco, CA (US)

(21) Appl. No.: 16/597,141

(22) Filed: Oct. 9, 2019

Related U.S. Application Data
(60) Provisional application No. 62/743,153, filed on Oct. 9, 2018.

(51) Int. Cl. C07K 16/28; G01N 33/50

(52) U.S. Cl. CPC C07K 16/2803; G01N 2500/00; G01N 33/5058

(57) ABSTRACT

The presently disclosed subject matter relates to methods and compositions for determining synapse formation, e.g., synapse formation associated with the activity of multispecific antibodies such as T cell-dependent bispecific antibodies.

### CROSS-REFERENCE TO RELATED APPLICATIONS

This application claims priority to U.S. Provisional Patent Application Serial No. 62/743,153, filed Oct. 9, 2018, the contents of which are incorporated by reference in their entirety.

### FIELD OF INVENTION

The presently disclosed subject matter relates to methods and systems for determining synapse formation, e.g., synapse formation associated with the activity of multispecific antibodies such as T cell-dependent bispecific antibodies.

### BACKGROUND

Multi specific antibodies, such as bispecific antibodies, are important as research tools, diagnostic tools and as therapeutics. This is due, in large part, to the fact that such antibodies can be selected to bind with high specificity and affinity to two or more antigens or two or more epitopes present on an antigen. For example, in the case of cancer therapeutics, multispecific antibodies can be used to target a cancer cell, e.g., by binding an antigen present on the cancer cell, to an immune cell to trigger an immune response. In addition, multispecific antibodies can be used as ligands for heterodimeric receptors that are normally activated by their cognate ligand when it binds to and promotes interaction between the components of the receptor.

Targeting tumor-associated cell surface antigen with therapeutic monoclonal antibodies (mAbs) or antibody-drug conjugates (ADCs) has been shown to be very effective for the treatment of hematological and solid tumor malignancies. These molecules often rely on one or combinations of the following mechanisms of action (MOA) to kill tumor cells: antibody-dependent cell-mediated cytotoxicity (ADCC), complement dependent cytotoxicity (CDC), receptor blockade, or internalization and intracellular release of conjugated cytotoxic drug. Despite the differences in MOA, maximizing/optimizing target engagement along with the consideration of pharmacokinetic properties of the therapeutic molecules has been an important driver for dose/regimen decision. Accordingly, there is a need for methods and systems for maximizing/optimizing mAbs and ADCs for treating cancer.

T cell-dependent bispecific molecules (e.g., bispecific T cell engager (BiTE) and T cell dependent bispecific antibody (TDB)), as a subset of multispecific antibodies, represent an emerging and promising class of therapeutic molecules for the treatment of cancer. Blinatumomab, a CD3xCD19 BiTE, has been proven effective for the treatment of a rare form of acute lymphoblastic leukemia and recently received an accelerated approval by the FDA (Bargou, R., E. Leo, et al. (2008) “Tumor regression in cancer patients by very low doses of a T cell-engaging antibody.” Science 321: 974-977). Multiple novel T cell-dependent bispecifics are also in clinical development and have shown promising preliminary result. Accordingly, there is a need for methods and systems for developing and screening novel multispecific molecules for therapeutic use. Multiple novel T cell-dependent bispecifics are also in clinical development and have shown promising preliminary result. (Budde, L E, et al. (2018) “Mosunetuzumab, a Full-Length Bispecific CD20/CD3 Antibody, Displays Clinical Activity in Relapsed/Refractory B-Cell Non-Hodgkin Lymphoma (NHL): Interim Safety and Efficacy Results from a Phase 1 Study”, Blood 132: 399).

### SUMMARY OF THE INVENTION

The presently disclosed subject matter provides methods and systems for determining synapse formation, e.g., by screening multispecific antibodies (such as T cell-dependent bispecific (TDB) antibodies). In certain embodiments, the methods relate to screening a multispecific antibody, e.g., a T cell-dependent bispecific antibody, that is capable of inducing cellular synapse formation. In certain embodiments, the method comprises (a) contacting a multispecific antibody that binds to a first antigen and a second antigen with a first cell expressing the first antigen and a second cell expressing the second antigen, wherein a cellular synapse is formed between the first cell and the second cell upon binding of the multispecific antibody to the first and second antigens and (b) measuring activation of the first cell by the cellular synapse, and detectable activation of the first cell indicates that the multispecific antibody is capable of inducing cellular synapse formation.

The presently disclosed subject matter further provides methods of detecting cellular synapse formation. In certain embodiments, the method comprises (a) contacting a multispecific antibody that binds to a first antigen and a second antigen with a first cell expressing the first antigen and a second cell expressing the second antigen, wherein a cellular synapse is formed between the first cell and the second cell upon binding of the multispecific antibody to the first and second antigens; and (b) measuring activation of the first cell by the cellular synapse, and wherein detectable activation of the first cell indicates cellular synapse formation.

In certain embodiments, the multispecific antibody is a bispecific antibody. In certain embodiments, measuring activation of the first cell comprises measuring at a biomarker indicative of activation. In certain embodiments, the biomarker is a cell surface molecule. In certain embodiments, the biomarker is selected from the group consisting of CD62L, CD69, CD154, and combinations thereof. In certain embodiments, the biomarker is the expression of CD62L. In certain embodiments, the first cell is a T cell or a cell derived from a T cell. In certain embodiments, the first cell has a deficient cytolytic ability upon activation. In certain embodiments, the first cell is a Jurkat cell. In certain embodiments, the first antigen is CD3.

In certain embodiments, the second antigen is a tumor antigen. In certain embodiments, the tumor antigen is selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B. In certain embodiments, the second cell is a B cell. In certain embodiments, the tumor antigen is selected from the group consisting of CD20, FcRH5, CD19, CD33, CD22, CD79A and CD79B.

In certain embodiments, measuring activation of the first cell comprises detecting a reporter that is induced upon the activation of the first cell. In certain embodiments, the reporter is a fluorescent or luminescent molecule.

In certain embodiments, the ratio of the first cell to the second cell is between about 1:10 and about 50:1. In certain embodiments, the ratio of the first cell to the second cell is between about 1:10 and about 10:1. In certain embodiments, the average expression of the second antigen on the second cell is at least about 1,000 molecules per cell. In certain embodiments, the average expression of the second antigen on the second cell is at least about 100,000 molecules per cell. In certain embodiments, the average distance between the first cell and the second cell is no more than about 0.3 mm. In certain embodiments, the average distance between the first cell and the second cell is no more than about 0.1 mm.

The presently disclosed subject matter is also directed to kits for determining cellular synapse formation, e.g., cellular synapse formation induced by a multispecific antibody that binds to a first antigen and a second antigen, where the first antigen is expressed by a first cell and the second antigen is expressed by a second cell. In certain embodiments, the kits of the present disclosure comprise (a) a first cell expressing the first antigen; (b) a second cell expressing the second antigen; and (c) means for measuring activation of the first cell. In certain embodiments, a cellular synapse is formed between the first cell and the second cell upon binding of the multispecific antibody to the first antigen and the second antigen. In certain embodiments, the cellular synapse formation activates the first cell.

The presently disclosed subject matter further provides systems for determining cellular synapse formation where the system comprises (a) a first cell expressing the first antigen; (b) a second cell expressing the second antigen; and (c) means for measuring activation of the first cell.

### BRIEF DESCRIPTION OF THE FIGURES

FIG. 1 depicts the structure of the cellular synapse model, describing the binding of T cell dependent bispecific antibody (TDB) to B lymphoma cell and T-cell, and the formation of cellular synapse.

FIGS. 2A-2B depict use of CD 69 and CD62L as biomarkers for T cell activation. FIG. 2A depicts Jurkat T cells incubated with BJAB B cells and CD20/CD3 TDB stained for CD69 and CD62L expression. FIG. 2B depicts that the percentage of T cells with increased CD69 or decreased CD62L was used to calculate % T cell activation opposite TDB concentration. Error bars indicate SEM.

FIGS. 3A-3B depict detection of T cell activation. FIG. 3A depicts that Jurkat T cells were incubated with BJAB B cells and CD20/CD3 TDB over a 24 hour time course. The percentage of activation as marked by CD69 increase or C62L decrease was calculated and plotted. FIG. 3B depicts that Jurkat T cells were incubated with BJAB B cells and CD20/CD3 TDB concentration titration over a 4 hour time course. The decrease in CD62L expression was used to calculate percentage T cell activation. T cell activation was plotted opposite TDB concentration. Error bars indicate SEM.

FIG. 4 depicts detection of CD4 and CD8 T cell activation. Human PMBCs were incubated with CD20/CD3 TDB for 4 hours. The percentage of CD4 and CD8 T cell activation measured by C62L decrease was calculated and plotted. Error bars indicate SEM.

FIG. 5 depicts predicted versus observed cellular synapse. Black circles represent observed cellular synapse percentage (number of T cell with CD62L T cell activation marker normalized to total number of T cell) at various effector:target (E:T) cell ratio and CD20/CD3 TDB concentration. Gray circles represent model-predicted cellular synapse percentage.

FIG. 6 depicts that the T cells are more likely to be activated when B cells had a higher expression level of the antigen CD20. B cell R is CD20, the expression levels are 1,200 per cell, 1,400 per cell or 122,000 per cell in the test samples.

FIG. 7 depicts a simulation of 500 T cells and 500 B cell in 1 μL.

FIG. 8 depicts simulations of intracellular distance between T cells and B cells.

FIG. 9 depicts T cell activation at conditions of different B cell antigen expression (1,200 per cell, 1,400 per cell or 122,000 per cell) and intracellular distance between T cells and B cells (distance (Dx) from 0.04 mm to 0.30 mm).

### DETAILED DESCRIPTION

1. Definitions

The term “antibody” herein is used in the broadest sense and encompasses various antibody structures, including but not limited to monoclonal antibodies, polyclonal antibodies, multispecific antibodies (e.g., bispecific antibodies and TDB antibodies), as well as antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. In certain embodiments, the antibody fragment is a Fab molecule. In certain embodiments, the antibody fragment is a F(ab′)₂ molecule.

The terms “full length antibody,” “intact antibody,” and “whole antibody” are used herein interchangeably to refer to an antibody having a structure substantially similar to a native antibody structure or having heavy chains that contain an Fc region as defined herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (C_{H}1, C_{H}2, and C_{H}3). Similarly, from N- to C-terminus, each light chain has a variable region (VL), also called a variable light domain or a light chain variable domain, followed by a constant light (CL) domain. The light chain of an antibody can be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

An “isolated” antibody or antibody fragment is one which has been separated from a component of its natural environment. An antibody or an antibody fragment can be purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “epitope,” as used herein, refers to a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny can be completely identical in nucleic acid content to a parent cell, or can contain mutations. Mutant progeny that have the same function or biological activity as screened or selected for in the originally transformed cell are included herein.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

2. Antibodies

The presently disclosed subject matter provides multispecific antibodies, e.g., bispecific antibodies and TDB antibodies, that can be evaluated by a screening method disclosed herein. A multispecific antibody, e.g., a bispecific antibody, of the present disclosure has at least two different binding specificities. See, e.g., U.S. Pat. Nos. 5,922,845 and 5,837,243; Zeilder (1999) J. Immunol. 163:1246-1252; Somasundaram (1999) Hum. Antibodies 9:47-54; Keler (1997) Cancer Res. 57:4008-4014. In certain embodiments, the multispecific antibodies encompasses by the present disclosure can bind to at least two different epitopes on a single antigen or bind to at least two epitopes that overlap on an antigen, e.g., a biepitopic antibody. Alternatively, in certain embodiments, the multispecific antibodies of the present disclosure can bind to at least two different antigens. The presently disclosed multispecific antibodies can be agonistic antibodies or antagonistic antibodies.

In certain embodiments, at least one antigen binding domain of the multispecific antibodies disclosed herein binds to one or more tumor antigens. Any tumor antigen can be used in the tumor-related embodiments described herein. The antigen can be, for example, expressed as a peptide or as an intact protein or portion thereof. The intact protein or a portion thereof can be native or mutagenized. Non-limiting examples of tumor antigens include HER2, LYPD1, LY6G6D, PMEL17, LY6E, EDAR, GFRA1, MRP4, RET, Steap1, TenB2, CD20, FcRH5, CD19, CD33, CD22, CD79A and CD79B. In certain embodiments, the tumor antigen is comprised in a B cell lymphoma. In certain embodiments, the tumor antigen is CD20, FcRH5, CD19, CD33, CD22, CD79A and CD79B.

In certain embodiments, at least one antigen binding domain of the multispecific antibody binds to one or more protein expressed on a cell, wherein the binding activates the cell. In certain embodiments, the cell is a T cell or a cell derived from T cell. In certain embodiments, the multispecific antibody binds to a receptor of a T cell or a cell derived from a T cell, wherein the binding can activate the cell. In certain embodiments, the multispecific antibody binds to CD3.

In certain embodiments, the multispecific antibody binds to a first antigen and a second antigen, wherein binding of the multispecific antibody to the first and second antigens activates the cell. In certain embodiments, the first antigen is a tumor antigen. In certain embodiments, the first antigen is CD3 and the second antigen is CD2O. In certain embodiments, the multispecific antibody is a bispecific antibody disclosed in International Publication NO. WO 2015/095392, which is incorporated herein by reference in its entirety.

In certain embodiments, the multispecific antibodies, e.g., bispecific antibodies and/or TDB antibodies, of the present disclosure comprise one or more antigen-binding polypeptides. For example, and not by way of limitation, a multispecific antibody of the present disclosure can include a first antigen-binding polypeptide and a second antigen-binding polypeptide. In certain embodiments, the first antigen-binding polypeptide and the second antigen-binding polypeptide have different binding specificities. In certain embodiments, the first antigen-binding polypeptide can bind to a first antigen and the second antigen-binding polypeptide can bind to a second antigen.

In certain embodiments, e.g., where a multispecific antibody of the present disclosure comprises a first antigen-binding polypeptide and a second antigen-binding polypeptide, the first antigen-binding polypeptide and second antigen-binding polypeptide can interact by one or more disulfide bridges. For example, and not by way of limitation, in certain embodiments, the hinge regions of the first and second antigen-binding polypeptides can interact by one or more disulfide bridges, e.g., by two disulfide bridges.

In certain embodiments, the multispecific, e.g., bispecific, antibodies include a heterodimerization domain within each of the antigen-binding polypeptides of the antibody, as disclosed herein. In certain embodiments, the CH3 domains of the first and second antigen-binding polypeptide of a disclosed multispecific antibody can be altered to promote heterodimerization of the first and second antigen-binding polypeptides. For example, and not by way of limitation, the first and/or second antigen-binding polypeptides can include one or more heterodimerization domains using knob-in-hole technology (see, e.g., U.S. Pat. Nos. 5,731,168 and 8,216,805, which are incorporated herein by reference in their entireties) to promote the association and/or interaction between the first antigen-binding polypeptide and the second antigen-binding polypeptide.

In certain embodiments, a multispecific antibody of the present disclosure does not include a light chain constant domain (CL). Alternatively, a multispecific antibody disclosed herein can include one or more CL domains. The presently disclosed subject matter further provides antagonistic and agonistic antibodies.

In certain embodiments, an antibody provided herein is an antibody fragment. Antibody fragments include, but are not limited, to F(ab′)_{2,} diabodies and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also PCT Application No. WO 93/16185; and U.S. Pat. Nos. 5,571,894 and 5,587,458.

Diabodies are antibody fragments with two antigen-binding sites that may be bivalent or bispecific. See, for example, EP Patent Application No. 404,097; PCT Application No. WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Additional non-limiting examples of antibody fragments include Fab, Fab′, Fab′-SH, Fv and scFv fragments. Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

For discussion of Fab and F(ab′)₂ fragments comprising salvage receptor binding epitope residues and having increased in vivo half-life, see U.S. Pat. No. 5,869,046.

In certain embodiments, the multispecific and bispecific antibodies provided herein are chimeric antibodies. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit or non-human primate, such as a monkey) and a human constant region. In certain embodiments, a chimeric antibody can be a “class switched” antibody in which the class or subclass has been changed from that of the parent antibody. Chimeric antibodies include antigen-binding fragments thereof.

In certain embodiments, a chimeric antibody is a humanized antibody. A non-human antibody can be humanized to reduce immunogenicity to humans, while retaining the specificity and affinity of the parental non-human antibody. A humanized antibody can include one or more variable domains in which hypervariable regions (HVRs), e.g., CDRs, or portions thereof, are derived from a non-human antibody, and FRs, or portions thereof, are derived from human antibody sequences. A humanized antibody optionally can also include at least a portion of a human constant region. In certain embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, the multispecific antibodies provided herein are human antibodies. Human antibodies can be produced using various techniques known in the art. Human antibodies are described, generally, in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals can be further modified, e.g., by combining with a different human constant region.

In certain embodiments, human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

In certain embodiments, human antibodies can also be generated by isolating Fv clone variable domain sequences selected from human-derived phage display libraries. Such variable domain sequences may then be combined with a desired human constant domain. Techniques for selecting human antibodies from antibody libraries are described below.

The presently disclosed subject matter also provides immunoconjugates, which include a multispecific antibody, e.g., a bispecific antibody, disclosed herein, conjugated to one or more cytotoxic agents, such as chemotherapeutic agents or drugs, growth inhibitory agents, proteins, peptides, toxins (e.g., protein toxins, enzymatically active toxins of bacterial, fungal, plant, or animal origin, or fragments thereof), or radioactive isotopes. For example, an antibody or antigen-binding portion of the disclosed subject matter can be functionally linked (e.g., by chemical coupling, genetic fusion, noncovalent association or otherwise) to one or more other binding molecules, such as another antibody, antibody fragment, peptide or binding mimetic.

3. Methods and Systems for Screening Antibodies

The multispecific antibodies of the presently disclosed subject matter can be identified, screened for, or their physical/chemical properties and/or biological activities characterized by the methods and systems provided herein.

T cell-dependent multispecific antibodies, for example, can activate effector T cells and targeting their cytolytic activity against target tumor cells. The mechanism of action of a T cell-dependent multispecific antibody, e.g., a TDB antibody, is dependent upon formation of cellular synapse. Therefore, in certain embodiments, selection of such a multispecific antibody can be based on a system and/or method that detects the antibody's ability of inducing cellular synapse formation.

In certain embodiments, the screening method comprises: (a) contacting a multispecific antibody that binds to a first antigen and a second antigen with a first cell (e.g., an effector cell) expressing the first antigen and a second cell (e.g., a target cell) expressing the second antigen, wherein a cellular synapse is formed between the first cell and the second cell upon binding of the multispecific antibody to the first and second antigens and (b) measuring activation of the first cell by the cellular synapse, wherein detectable activation of the first cell indicates that the multispecific antibody is capable of inducing cellular synapse formation. In certain embodiments, the multispecific antibody is a bispecific antibody.

In certain embodiments, the first cell (e.g., effector cell) is a T cell or a cell derived from a T cell. Non-limiting examples of T cells include helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells.

In certain embodiments, the first cell is engineered so that it is cytolytically deficient upon activation. Non-limiting examples of such engineering include, but are not limited to, deletion or disruption of one or more genes involved in cytolytic activity, e.g., perforin and granzyme, any antitumor cytokine (e.g., IL-2, IFNγ and TNFα) and/or one or more genes required for the expression of these genes. In certain embodiments, the first cell is an immortalized cell. In certain embodiments, the first cell is a Jurkat cell.

In certain embodiments, the first cell expresses a first antigen. In certain embodiments, binding of the multispecific antibody to the first antigen is capable of activating the first cell. In certain embodiments, the antigen is a receptor. In certain embodiments, the antigen is in a biological complex. For example, and not by way of limitation, the receptor is present within a biological complex, e.g., in a complex with one or more co-receptors and/or proteins. In certain embodiments, the first antigen is a component of a CD3 receptor.

In certain embodiments, measuring activation of the first cell comprises measuring a biomarker indicative of activation. In certain embodiments, the biomarker is a cell surface molecule, the quantity of which changes upon activation of the first cell. Changes of a cell surface molecule can be determined by any assay known in the art and disclosed herein. For example, and not by way of limitation, a cell surface molecule can be measured by enzyme-linked immunosorbent assay (ELISA) or by flow cytometry, e.g., fluorescence activated cell sorting (FACS) using an antibody that targets the cell surface molecule. In certain embodiments, the biomarker is selected from the group consisting of CD62L, CD69, and a combination thereof. In certain embodiments, the biomarker is the expression of CD62L.

In certain embodiments, the second cell (e.g., target cell) is a tumor cell or a cell expressing a tumor antigen. In certain embodiments the second antigen is a tumor antigen. In certain embodiments the tumor antigen is selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B. In certain embodiments, the second antigen is endogenous to the second cell. In certain embodiments, the second cell is a B cell. In certain embodiments, the tumor antigen is selected from the group consisting of CD20, FcRH5, CD19, CD33, CD22, CD79A and CD79B

Genetic modification of a cell (e.g., modification of a first cell and/or a second cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors can be used as well.

In certain embodiments, the activation of the first cell can be determined by analyzing whether a signaling pathway is associated with the activation of the first cell. In certain embodiments, measuring activation of the first cell comprises detecting a reporter that is induced upon the activation of the first cell. For example, but not by way of limitation, the activation can be determined by using an in vitro reporter-based assay, e.g., a luciferase assay, where the activation of the first antigen, e.g., a receptor, results in the expression of a reporter, e.g., luciferase or a fluorescence protein, e.g., GFP or RFP. In certain embodiments, the reporter is expressed from a construct comprising a promoter that is activated upon activation of the first cell. Non-limiting examples of the promoters include CD69 promoter and IL-2 promoter.

In certain embodiments, the formation of the cellular synapse and/or the activation of the first cell is affected by the ratio between the first cell and the second cell. In certain embodiments, the ratio of the first cell to the second cell is between about 1:1000 and about 1000:1, between about 1:500 and about 500:1, between about 1:200 and about 200:1, between about 1:100 and about 100:1, between about 1:50 and about 50:1, between about 1:40 and about 40:1, between about 1:30 and about 30:1, between about 1:20 and about 20:1, between about 1:10 and about 10:1, between about 1:5 and about 5:1, between about 1:4 and about 4:1, between about 1:3 and about 3:1, or between about 1:2 and about 2:1. In certain embodiments, the ratio of the first cell to the second cell is between about 1:10 and about 50:1. In certain embodiments, the ratio of the first cell to the second cell is between about 1:10 and about 10:1. In certain embodiments, the ratio of the first cell to the second cell is about 1:1000, about 1:500, about 1:400, about 1:300, about 1:200, about 1:100, about 1:50, about 1:40, about 1:30, about 1:20, about 1:10, about 1:9, about 1:8, about 1:7, about 1:6, about 1:5, about 1:3, about 1:2, about 1:1, about 2:1, about 3:1, about 4:1, about 5:1, about 6:1, about 7:1, about 8:1, about 9:1, about 10:1, about 20:1, about 30:1, about 40:1, about 50:1, about 100:1, about 200:1, about 300:1, about 400:1, about 500:1, or about 1000:1.

In certain embodiments, the formation of the cellular synapse and/or the activation of the first cell is affected by the expression of the second antigen on the second cell. In certain embodiments, the average expression of the second antigen on the second cell is at least about 10 molecules per cell, at least about 100 molecules per cell, at least about 1,000 molecules per cell, at least about 2,000 molecules per cell, at least about 3,000 molecules per cell, at least about 4,000 molecules per cell, at least about 5,000 molecules per cell, at least about 6,000 molecules per cell, at least about 7,000 molecules per cell, at least about 8,000 molecules per cell, at least about 9,000 molecules per cell, at least about 10,000 molecules per cell, at least about 15,000 molecules per cell, at least about 20,000 molecules per cell, at least about 30,000 molecules per cell, at least about 40,000 molecules per cell, at least about 50,000 molecules per cell, at least about 60,000 molecules per cell, at least about 70,000 molecules per cell, at least about 80,000 molecules per cell, at least about 90,000 molecules per cell, at least about 100,000 molecules per cell, at least about 110,000 molecules per cell, at least about 120,000 molecules per cell, at least about 130,000 molecules per cell, at least about 140,000 molecules per cell, at least about 150,000 molecules per cell, at least about 160,000 molecules per cell, at least about 170,000 molecules per cell, at least about 180,000 molecules per cell, at least about 190,000 molecules per cell, at least about 200,000 molecules per cell, at least about 300,000 molecules per cell, at least about 400,000 molecules per cell, at least about 30,000 molecules per cell, at least about 500,000 molecules per cell, or at least about 1000,000 molecules per cell. In certain embodiments, the average expression of the second antigen on the second cell is between about 10 to about 100 molecules per cell, between about 100 to about 1,000 molecules per cell, between about 100 to about 10,000 molecules per cell, between about 1,000 to about 100,000 molecules per cell, between about 1,000 to about 200,000 molecules per cell, between about 1000 to about 300,00 molecules per cell, between about 10,000 to about 100,000 molecules per cell, between about 10,000 to about 200,000 molecules per cell, or between about 10,000 to about 500,000 molecules per cell.

In certain embodiments, the formation of the cellular synapse and/or the activation of the first cell is affected by the density of or average distance between the first cell and the second cell.

Intracellular distances can be determined by any methods know in the art. For example, method to calculate distance between the first cells and the second cells can comprise: using a software to simulate experimental cell numbers with random x,y,z coordinates within a cube having a size of e.g., 1 μL (1 mm³), determining the average distance between each cell and 6 closes cells, and determining the overall average distance to reach a final average distance value. In certain embodiments, the average distance between the first cell and the second cell is no more than about 10 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, no more than about 0.2 mm, no more than about 0.1 mm, no more than about 0.09 mm, no more than about 0.08 mm, no more than about 0.07 mm, no more than about 0.06 mm, no more than about 0.05 mm, no more than about 0.04 mm, no more than about 0.03 mm, no more than about 0.02 mm, no more than about 0.01 mm, no more than about 0.005 mm, no more than about 0.001 mm, no more than about 0.0005 mm, or no more than about 0.0001 mm. In certain embodiments, the average distance between the first cell and the second cell is between about 0.0001 mm and about 100 mm, between about 0.001 mm and about 10 mm, between about 0.005 mm and about 5 mm, between about 0.01 mm and about 1 mm, between about 0.02 mm and about 1 mm, between about 0.03 mm and about 1 mm, between about 0.04 mm and about 1 mm, between about 0.05 mm and about 1 mm, or between about 0.01 mm and about 0.5 mm.

4. System and Kits.

The presently disclosed subject matter further relates to systems and kits. In certain embodiments, a system/kit disclosed herein can be used to determine cellular synapse formation of a multispecific antibody that binds to a first antigen and a second antigen. In certain embodiments, the system/kit comprises (a) a first cell expressing the first antigen; (b) a second cell expressing the second antigen; and (c) means for measuring activation of the first cell. In certain embodiments, a cellular synapse is formed between the first cell and the second cell upon binding of the multispecific antibody to the first antigen and the second antigen. In certain embodiments, the cellular synapse formation activates the first cell.

In certain embodiments, the system/kit includes a container and a label or package insert on or associated with the container. The containers can be formed from a variety of materials such as glass or plastic. The container can hold a composition which is by itself or combined with another composition.

If desired, the system/kit can be provided together with instructions for any methods disclose herein. The instructions can generally include information about the use of the composition for performing the methods. The instructions may be printed directly on the container (when present), or as a label applied to the container, or as a separate sheet, pamphlet, card, or folder supplied in or with the container.

The following examples are merely illustrative of the presently disclosed subject matter and should not be considered as limitations in any way.

5. Exemplary Non-Limiting Embodiments.

A. A method of detecting cellular synapse formation comprising: contacting a multispecific antibody capable of binding to a first antigen and a second antigen with a first cell expressing the first antigen and a second cell expressing the second antigen, wherein a cellular synapse is formed between the first cell and the second cell upon binding of the multispecific antibody to the first and second antigens; and measuring activation of the first cell, wherein activation of the first cell indicates cellular synapse formation.

A1. A method of determining the activity of a multispecific antibody capable of inducing cellular synapse formation comprising: contacting the multispecific antibody that binds to a first antigen and a second antigen with a first cell expressing the first antigen and a second cell expressing the second antigen, wherein a cellular synapse is formed between the first cell and the second cell upon binding of the multispecific antibody to the first and second antigens; and measuring activation of the first cell by the cellular synapse, wherein detectable activation of the first cell indicates that the multispecific antibody is capable of inducing cellular synapse formation.

A2. The method of A or A1, wherein measuring activation of the first cell comprising measuring at least one biomarker indicative of activation.

A3. The method of A2, wherein the at least one biomarker is a cell surface molecule.

A4. The method of A3, wherein the at least one biomarker is selected from the group consisting of CD62L, CD69, and a combination thereof.

A5. The method of A4, wherein the at least one biomarker is the expression of CD62L.

A6. The method of any one of A-A5, wherein the first antigen is CD3.

A7. The method of any one of A-A6, wherein the first cell is a T cell or a cell derived from a T cell.

A8. The method of A7, wherein the first cell is cytolytically deficient upon activation.

A9. The method of A8, wherein the first cell is a Jurkat cell.

A10. The method of any one of A-A9, wherein the second antigen is a tumor antigen.

A11. The method of A10 wherein the tumor antigen is selected from the group consisting of HER2, LYPD1, LY6G6D, PMEL17, LY6E, EDAR, GFRA1, MRP4, RET, Steapl, TenB2, CD20, FcRHS, CD19, CD33, CD22, CD79A and CD79B.

A12. The method of any one of A-A11, wherein the second cell is a B cell.

A13. The method of A12, wherein the tumor antigen is selected from the group consisting of CD20, FcRHS, CD19, CD33, CD22, CD79A and CD79B.

A14. The method of any one of A, A1, and A6-A13, wherein measuring activation of the first cell comprises detecting a reporter that is induced upon the activation of the first cell.

A15. The method of A14, wherein the reporter is a fluorescent or luminescent molecule.

A16. The method of any A-A15, wherein the ratio of the first cell to the second cell is between about 1:10 and about 50:1.

A17. The method of A16, wherein the ratio of the first cell to the second cell is between about 1:10 and about 10:1.

A18. The method of any one of A-A17, wherein the average expression of the second antigen on the second cell is at least about 1,000 molecules per cell.

A19. The method of A18, wherein the average expression of the second antigen on the second cell is at least about 10,000 molecules per cell.

A20. The method of any one of A-A19, wherein the average distance between the first cell and the second cell is no more than about 0.3 mm.

A21. The method of A20, wherein the average distance between the first cell and the second cell is no more than about 0.1 mm.

A22. The method of any one of A-A21, wherein the multispecific antibody is a bispecific antibody.

B. A kit for determining cellular synapse formation of a multispecific antibody that binds to a first antigen and a second antigen, comprising: a first cell expressing the first antigen; a second cell expressing the second antigen; and a means for measuring activation of the first cell.

B 1. The kit of B, wherein a cellular synapse is formed between the first cell and the second cell upon binding of the bispecific antibody to the first antigen and the second antigen.

B2. The kit of B1, wherein the cellular synapse formation activates the first cell.

B3. The kit of any one of B-B2, wherein the means for measuring activation of the first cell comprises measuring at least one biomarker indicative of activation.

B4. The kit of B3, wherein the at least one biomarker is a cell surface molecule.

B5. The kit of B4, wherein the at least one biomarker is selected from the group consisting of the expression of CD62L, CD69, and a combination thereof.

B6. The kit of B5, wherein the at least one biomarker comprises expression of CD62L.

B7. The kit of any one of B-B5, wherein the first antigen is CD3.

B8. The kit of any one of B-B5, wherein the first cell is a T cell or a cell derived from a T cell.

B9. The kit of B8, wherein the first cell is cytolytically deficient upon activation.

B10. The kit of B9, wherein the first cell is a Jurkat cell.

B11. The kit of any one of B-B9, wherein the second antigen is a tumor antigen.

B12. The kit of B11, wherein the tumor antigen is selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B.

B13. The kit of any one of B-B12, wherein the second cell is a B cell.

B14. The kit of B13, wherein the tumor antigen is selected from the group consisting of CD20, FcRH5, CD19, CD33, CD22, CD79A and CD79B.

B15. The kit of any one of B-B2 and B7-B14, wherein the means for measuring activation of the first cell comprises a reporter gene in the first cell, where expression of the reporter gene is induced upon the activation of the first cell.

B16. The kit of B15, wherein the reporter gene expresses a fluorescent or luminescent molecule.

B17. The kit of any one of claims B-B16, wherein the ratio of the first cell to the second cell is between about 1:10 and about 50:1.

B18. The kit of B17, wherein the ratio of the first cell to the second cell is between about 1:10 and about 10:1.

B19. The kit of any one of B-B18, wherein the average expression of the second antigen on the second cell is at least about 1,000 molecules per cell.

B20. The kit of B19, wherein the average expression of the second antigen on the second cell is at least about 10,000 molecules per cell.

B21. The kit of any one of B-B20, wherein the average distance between the first cell and the second cell is no more than about 0.3 mm.

B22. The kit of B21, wherein the average distance between the first cell and the second cell is no more than about 0.1 mm.

B23. The kit of any one of B-B22, wherein the multispecific antibody is a bispecific antibody.

C. A system for determining cellular synapse formation of a multispecific antibody that binds to a first antigen and a second antigen, comprising: a first cell expressing the first antigen; a second cell expressing the second antigen; and a means for measuring activation of the first cell.

C1. The system of C, wherein a cellular synapse is formed between the first cell and the second cell upon binding of the multispecific antibody to the first antigen and the second antigen.

C2. The system of C1, wherein the cellular synapse formation activates the first cell.

C3. The system of any one of C-C2, wherein the means for measuring activation of the first cell comprises at least one biomarker indicative of activation.

C4. The system of C3, wherein the at least one biomarker is a cell surface molecule.

C5. The system of C4, wherein the at least one biomarker is selected from the group consisting of expression of CD62L, CD69, and a combination thereof.

C6. The system of C5, wherein the at least one biomarker comprises expression of CD62L.

C7. The system of any one of C-C6, wherein the first antigen is CD3.

C8. The system of any one of C-C7, wherein the first cell is a T cell or a cell derived from a T cell.

C9. The system of C8, wherein the first cell is cytolytically deficient upon activation.

C10. The system of C9, wherein the first cell is a Jurkat cell.

C11. The system of any one of C-C10, wherein the second antigen is a tumor antigen.

C12. The system of C11, wherein the tumor antigen is selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B.

C13. The system of any one of C-C12, wherein the second cell is a B cell.

C14. The system of C13, wherein the tumor antigen is selected from the group consisting of CD20, FcRH5, CD19, CD33, CD22, CD79A and CD79B.

C15. The system of any one of C-C2 and C13-C14, wherein the means for measuring activation of the first cell comprises a reporter gene in the first cell, where expression of the reporter gene is induced upon the activation of the first cell.

C16. The system of C15, wherein the reporter gene expresses a fluorescent or luminescent molecule.

C17. The system of any one of C-C16, wherein the ratio of the first cell to the second cell is between about 1:10 and about 50:1.

C18. The system of C17, wherein the ratio of the first cell to the second cell is between about 1:10 and about 10:1.

C19. The system of any one of C-C18, wherein the average expression of the second antigen on the second cell is at least about 1,000 molecules per cell.

C20. The system of C19, wherein the average expression of the second antigen on the second cell is at least about 10,000 molecules per cell.

C21. The system of any one of C-C20, wherein the average distance between the first cell and the second cell is no more than about 0.3 mm.

C22. The system of C21, wherein the average distance between the first cell and the second cell is no more than about 0.1 mm.

C23. The system of any one of C-C22, wherein the multispecific antibody is a bispecific antibody.

### EXAMPLES

The following are examples of the methods and compositions of the present disclosure. It is understood that various other embodiments can be practiced, given the general description provided above.

### Example 1—Development of a Model and In Vitro Assay System for Cellular Synapse Formation by T Cell Dependent Bispecific Molecule

Unlike MOA of typical therapeutic mAb or ADC, T cell-dependent bispecific molecules work by activating effector T cells and targeting their cytolytic activity against target tumor cells (Staerz, U. D., O. Kanagawa, and M. J. Bevan.(1985) “Hybrid antibodies can target sites for attach by T cells.” Nature 314:628-631). The MOA of the bispecific molecule is dependent upon simultaneous engagement of both the tumor cell and CD3-expressing T cell (Baeuerle, P. A., C. Reihardt, and Kufer P. (2008) “BiTE: a new class of antibodies that recruit T-cells.” Drugs of the Future 33(2): 137-147). Co-engagement of both cells leads to formation of cellular synapse, which then induces polyclonal activation of T cell through the T cell receptor (TCR) and release of perforin and granzyme for the lysis of the target tumor cell (Bauerle, 2008, Chen, 2016). Several factors could potential impact of the formation of cellular synapse, including concentration of T cell-dependent bispecific molecules, cell density of target tumor cells, cell density of CD3-expressing T cells, binding affinities to target and CD3, as well as target and CD3 expression level. The unique MOA and the multiple determinants on cellular synapse formations can be assessed using an integrated analysis and mechanistic model to systemically evaluate the individual effect of various factors.

In this example, an in vitro assay was established using an early marker of T cell activation as a surrogate for cellular synapse formation. Data derived from the in vitro assay was used to develop a mechanism-based model to simultaneously assess the effect of various factors on cellular synapse formation. The modeling framework can guide the rational design and development of T cell-dependent bispecific molecules, as well as other multispecific antibodies more generally.

Materials & Methods

Reagents: Cells and Test Antibodies

B lymphoma cell lines including BJAB, Pfeiffer, and SUDHL-8, as well as Jurkat, a human lymphoblast cell line derived from acute lymphocytic leukemia, were obtained from the American Type Culture Collection (Manassas, Va.). These cell lines were maintained in Roswell Park Memorial Institute (RPMI) medium 1640 (Corning, Tewksbury, Mass.) supplemented with 10% fetal bovine serum (FBS: Hyclone, Logan, UT), 25 mM HEPES (Corning, Tewksbury, Mass.), 1% Glutamax (Gibco, Carlsbad, Calif.), and 1% penicillin/streptomycin (Gibco, Carlsbad, Calif.).

Anti-CD20/CD3 TDB is a humanized full-length IgG1 knob-in hole bi-specific antibody (Speiss, 2013). All antibodies were manufactured from engineered Chinese hamster ovary (CHO) cell lines at Genentech, Inc.

Cellular Synapse Assay with Jurkat Effector Cells

Effector T cells (Jurkat) were incubated with CD20-expressing target cells (BJAB/Pfeiffer/SUDHL8) at an effector to target ratio of 50:1, 10:1, 1:1, or 1:10. Effector and target cells were diluted in assay media (RPMI-1640, 10% FBS, 25 mM HEPES, 1% Glutamax, 1% penicillin/streptomycin). 50 uL of each cell type at the test concentration was seeded in a 96 well U-bottom plate (Falcon, Corning, N.Y.). TDB test antibody was diluted in assay media starting from 1 mg/mL followed by 10 7.5-fold serial dilutions and added to the cells. The reaction was incubated for 0-24 hrs at 37° C., 5% CO2. After incubation, the plate was transferred to ice to stop the reaction. The cells were washed 3 times with 200 uL of FACs buffer (PBS, 2% FBS, 0.02% azide) by centrifugation at 1200 RPM for 5 min at 4° C. to remove unbound antibody.

The cells were stained for CD19 expression on B cells (APC anti-human CD19, BioLegend, San Diego, Calif.) as well as T cell activation markers CD62L and CD69 (PE anti-human CD62L, FITC anti-human CD69, BD Biosciences, San Jose, Calif.) for 30 minutes on ice. After staining, cells were washed 3 times with 200 uL of FACs buffer and fixed in 4% paraformaldehyde for 10 min at 4° C. The cells were analyzed on a flow cytometer (BD Biosciences FACSCanto IVD 10, San Jose, Calif.). CD19-positive cells were gated as target cells and CD19-negative cells were gated as effector cells.

The average number of fluorescent CD62L or CD69 effector cells was analyzed with Flow Jo software (Treestar, Ashland, Oreg.). The baseline percentage was determined using the control (no TDB antibody) condition. The change in the percentage of CD62L or CD69 positive T cells was plotted opposite the TDB test antibody concentration (GrapPad Prism, La Jolla, Calif.).

Cellular Synapse Assay with Peripheral Blood Mononuclear cells (PBMCs)

PBMCs were isolated from fresh blood of healthy donors by density gradient centrifugation using a Uni-Sep blood separation tube (Accurate Chemical & Scientific, Westbury, N.Y.) following the manufacturer's instructions. The mononuclear cells from the interface were collected, washed twice with assay media (RPMI-1640, 10% FBS, 25 mM HEPES, 1% Glutamax, 1% penicillin/streptomycin). PBMCs were diluted in the assay media in the same volume as that of the blood from which they were isolated in order to maintain the physiological count of cells.100 uL of PBMCs were seeded in each well in a 96 well U-bottom plate (Falcon, Corning, N.Y.). TDB test antibody was diluted in assay media starting from 1 mg/mL followed by 10 5-fold serial dilutions and added to the cells. PBMCs with antibody were incubated for 4hrs at 37° C., 5% CO2. After incubation, the plate was transferred to ice to stop the reaction. The cells were washed 3 times with 200 uL of FACS Buffer (PBS, 2% FBS, 0.02% azide) by centrifugation at 1200 RPM for 5 min at 4° C. to remove unbound antibody. The cells were stained for B cell surface antigens (CD19, CD40), T cell surface antigens (CD4, CD8) and the T cell activation marker (CD62L) using the following antibodies: PECy7 anti-human CD19 (BioLegend), Brilliant Violet 510 Anti-Human CD4 (BioLegend), APC/Cy7 anti-human CD8 (BioLegend), and PE anti-human CD62L antibody (BD Biosciences) for 30 minutes on ice. After staining, cells were washed 3 times with 200 uL of FACS buffer (PBS, 2% FBS, 0.02% azide) and fixed in 4% paraformaldehyde for 10 min at 4° C.

The cells were analyzed on a flow cytometer (BD Biosciences FACSCanto IVD 10, San Jose, Calif.). CD19 positive cells were gated as B cells and CD4 positive and CD8 positive cells were gated as T cells. Activation of T cells causes shedding of L-selectin (CD62L). CD62L fluorescence on T cells was calculated using Flow Jo. The baseline fluorescence was determined by gating on the control (no antibody) population and normalized across multiple runs. Dose response curves were plotted in GraphPad Prism (LaJolla, Calif.).

Model Development

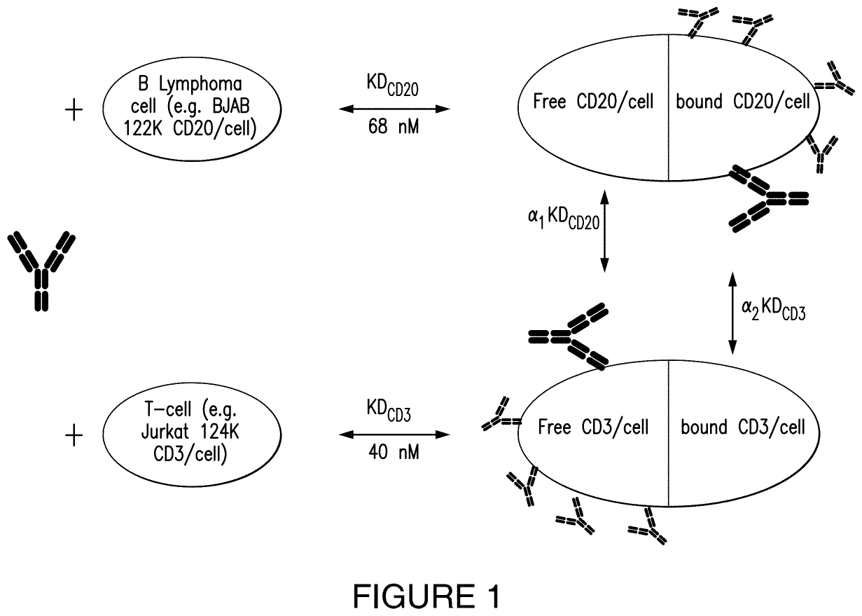

A mathematical model based on a series of ordinary differential equations was developed to describe formation of TDB synapse (FIG. 1), which describe the sequential bindings between CD20/CD3 TDB, tumor antigen CD20, and CD3 receptor in T cells. The binding affinity (KD) values of the CD20/CD3 TDB to CD20 and CD3 were 68 nM and 40 nM, respectively. In the current modeling exercise, the Koff values of CD20/CD3 TDB to CD20 or CD3 were derived using the equation koff=KD*kon under the assumption that kon values were 0.001 1/(nM×second) to ensure that binding equilibrium would be achieved under experimental conditions.

The synapse formation was proposed with the following assumption and stepwise approximation: 1) total amounts of cell-bound CD20 and CD3 are evenly distributed in a well-stirred system; 2) TDB first bind to CD20 or CD3 in a 1:1 ratio, and the bindings are independent events (Equations 1-5); 3) the TDB-bound CD20 and CD3 would then bind to unbound CD3 and CD20, respectively, to form tri-molecule synapse (Equations 6-10); 4) The relationship between tr-molecular synapse and cellular synapse were described by a Emax model (Equations 11); 5) the average distance of the six closest target cells (i.e. B lymphoma cells) to each T-cell was derived to account for the effects of cell density and relative cell density between target cell to T-cell on cellular synapse formation (See the next section).

d(Drug_free)/dt=−konCD20*CD20_free*Drug_free−konCD3*CD3_free*Drug_free+koffCD20*DrugCD20+koffCD3*DrugCD3 (1)

d(CD20_free)/dt=−konCD20*CD20_free*Drug_free+koffCD20*DrugCD20 (2)

d(CD3_free)/dt=−konCD3*CD3_free*Drug_free+koffCD3*DrugCD3 (3)

d(DrugCD20)/dt=konCD20*CD20_free*Drug_free−koffCD20*DrugCD20 (4)

d(DrugCD3)/dt=konCD3*CD3_free*Drug_free−koffCD3*DrugCD3 (5)

Drug free, CD20 free, and CD3_free represent unbound (or free) CD20/CD3 TDB, CD20, and CD3, respectively. DrugCD20 and DrugCD3 represent TDB-bound CD20 and TDB-bound CD3, respectively.

CD20_FPC=(CD20_free/CD20B0)*CD20_KCell (6)

CD20_BPC=(DCD20/CD20B0)*CD20_KCell (7)

CD3_FPC=(CD3F/CD3T0)*CD3_KCell (8)

CD3_BPC=(DCD3/CD3T0)*CD3_KCell (9)

Synapse_{M}=CD20_FPC*CD3_BPC/(α1*KDCD20)+CD20_BPC *CD3_FPC/(α2*KDCD3) (10)

CD20 FPC and CD20 BPC represent free and TDB-bound CD20 receptors/B cell, respectively. CD3 FPC and CD3 BPC represent free and TDB-bound CD3 receptors/T-cell, respectively. Synapse_{M} represents tri-molecular synapse. α represents scaling factor for KD.

Synapse_{C}=Emax*Synapse_{M}/(EC50+Synapse_{M}) (11)

Synapse_{C} represents cellular synapse.

Calculation of Intercellular Distance Between T-Cell and B Lymphoma Cell

Position of the T-cell and B lymphoma cell within a cube of 1 mm³ (or 1 uL) in size were simulated with random X-, Y-, and Z-coordinates according to the experimental conditions (Table 1) (R version 3.5.1 software). For each T-cell in the cube (n=500-2000), the average distance of the six closest target cells (i.e. B lymphoma cells) were calculated followed by averaging the distance for all T-cell in the cube (Table 2).

TABLE 1
Experimental conditions used to simulate position of T-cell and B lymphoma cells

| | T | B | T | B | Total | |
|---|---|---|---|---|---|---|
| Incubation | Cell Density | Cell Density | Cell Density | Cell Density | Cell Density | |
| Set | (cells/mL) | (cells/mL) | (cells/uL) | (cells/uL) | (cells/uL) | % B Cells |
| 1 | 500000 | 500000 | 500 | 500 | 1000 | 50.0 |
| 2 | 1000000 | 100000 | 1000 | 100 | 1100 | 9.09 |
| 3 | 1000000 | 200000 | 1000 | 200 | 1200 | 16.7 |
| 4 | 1000000 | 1000000 | 1000 | 1000 | 2000 | 50.0 |
| 5 | 1000000 | 10000000 | 1000 | 10000 | 11000 | 90.9 |
| 6 | 2000000 | 40000 | 2000 | 40 | 2040 | 1.96 |

TABLE 2
Average distance of B lymphoma cell to T-cell

| | | | Intracellular |
|---|---|---|---|
| Incubation | T Cell Density | B Cell Density | Distance |
| Set | (cell count/mL) | (cell count/mL) | (DX; mm) |
| 1 | 500000 | 500000 | 0.117 |
| 2 | 1000000 | 100000 | 0.207 |
| 3 | 1000000 | 200000 | 0.164 |
| 4 | 1000000 | 1000000 | 0.0910 |
| 5 | 1000000 | 10000000 | 0.0412 |
| 6 | 2000000 | 40000 | 0.299 |

This overall average distance (DX; mm) was then incorporated to Equations 11 to account for the effects of cell concentrations and ratio of target cell to T-cell on cellular synapse formation (Equations 12-15). The parameter estimates of final model were presented in Table 3.

Synapse_{C}=Emax*Synapse_{M}/(EC50+Synapse_{M}) (11)

Emax=Emax_{DX}−Emax_{DX}*DX\hat{ }GAM_{EmaxDX}/(EC50_{EmaxDX}\hat{ }GAM_{EmaxDX}+DX\hat{ }GAM_{EmaxDX}) (12)

Emax_{DX}=EMax_{EmaxDX,CD20}*CD20_Kcell/(EC50_{EmaxDX,CD20}+CD20_Kcell) (13)

EC50=Emax_{EC50DX}*DX/(EC50_{EC50DX}+DX) (14)

Emax_{EC50DX}=Emax_{EC50DX,CD20}*CD20_Kcell\hat{ }GAM_{EC50DX,CD20}/(EC50_{EC50DX,CD20}\hat{ }GAM_{EC50DX,CD20}+CD20_Kcell\hat{ }GAM_{EC50DX,CD20}) (15)

CD20_Kcell represents CD20 expression level per cell (receptor per cell).

TABLE 3
Estimated parameters of the model by fitting the in vitro cellular synapse data.

| Parameter | | | Units | Value | Description |
|---|---|---|---|---|---|
| Emax | Emax_{EmaxDx} | Emax_{EmaxDX, CD20} | % cellular | 57.7 | Maximum % cellular synapse |
| | | | synaspe | | |
| | | EC50_{EmaxDX, CD20} | CD20 | 0.690 | CD20 expression at 50% of |
| | | | receptors/B cell | | Emax_{EMmaxDX} |
| | | | (×1000) | | |
| | EC50_{EmaxDX} | | mm | 0.245 | Intracellular distance at 50% |
| | | | | | of Emax_{EmaxDX} |
| | GAM_{EmaxDX} | | — | 4.32 | Hill coefficient |
| EC50 | Emax_{EC50DX} | Emax_{EC50DX, CD20} | nM | 1.99 | Synapse concentration at 50% |
| | | | | | of Emax |
| | | EC50_{EC50DX, CD20} | CD20 | 2.86 | CD20 expression at 50% of |
| | | | receptors/B cell | | Emax_{EC50DX, CD20} |
| | | | (×1000) | | |
| | | GAM_{EC50DX, CD20} | — | 3.00 | Hill coefficient |
| | EC50_{EC50DX} | | mm | 0.463 | Intracellular distance at 50% |
| | | | | | maximum % T-cell activation |
| α₁ | | | — | 4.40 | Scaling factor on KD_{CD20} |
| α₂ | | | — | 1 | Scaling factor on KD_{CD3} |

Results

The mechanism of action of T cell dependent bispecific molecules has been well defined (Sun, 2015). The arm specific for the target antigen engages the cancer cell and the other arm engages the T cell simultaneously to induce polyclonal T cell activation. Activation of the T cell leads to release of perforin and granzyme which lyses the cancer cell. Therefore, the driving step in the TDB mechanism of action (MOA) is the binding of the TDB molecule to both target and effector T cell, forming the cellular synapse (Staerz, 1985). T cell activation is the most proximal event following cellular synapse formation, thus T cell activation markers can be used to approximate synapse formation.

To develop an in vitro assay to detect TDB-dependent synapse formation it can be useful to keep the balance of target and effector cells constant. Therefore, effector cells that activate but do no lyse target cells were employed. Jurkat T cells were used to develop the in vitro system as an alternative T cell source. Jurkat T cells activate similar to primary T cells, but do not release perforin and granzyme. CD20-expressing BJAB B lymphoma cells were used as the target cells. For initial assay development the cells were combined at a 1:1 effector to target ratio and were incubated together in the presence of CD20/CD3 TDB for four hours at 37° C.

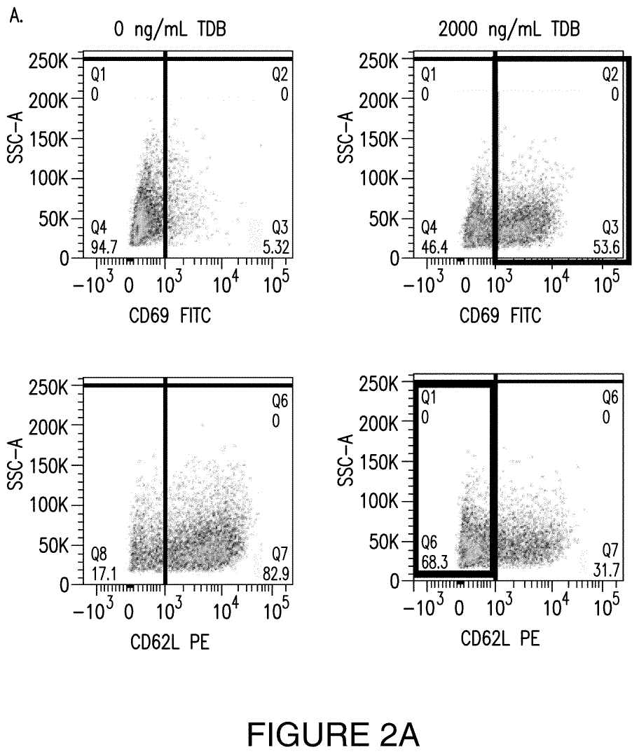

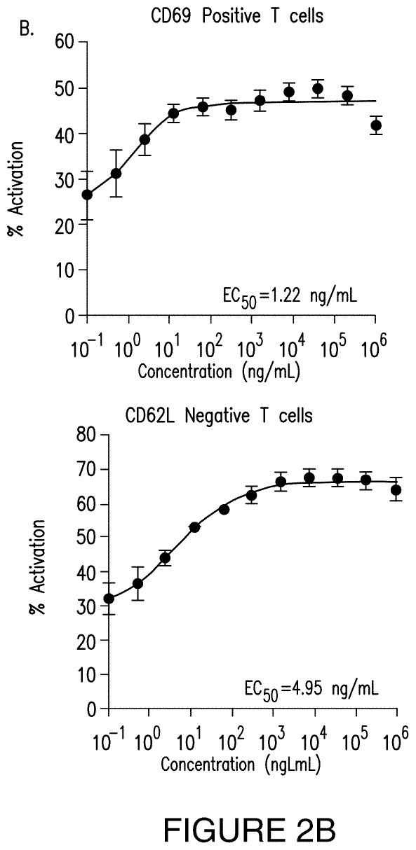

After incubation the cells were stained for CD19, CD69, and CD62L expression. CD19 expression was used to differentiate the target and CD3 T cells. CD62L shedding and CD69 upregulation from the surface of T cell are known T cell activation markers (Chao, C., R. Jensen, et al. (1997) “Mechanisms of L-Selectin Regulation by Activated T cells.” J Immunol 159:1686-1694; and Shipkova, M., E. Wieland. (2012) “Surface markers of lymphocyte activation and markers of cell proliferation.” Clinic Chimica Acta 413:1338-1349). Therefore, activated T cells were measured as CD69 positive or CD62L negative (FIG. 2A). The percentage of activated T cells was calculated and plotted against the TDB concentration (FIG. 2B). Both CD62L and CD69 showed similar patterns of activation (FIG. 2B). TDB-dependent CD69 activation had an EC50 of 1.22 ng/mL, while TDB-dependent CD62L-decrease had an EC50 of 4.95 ng/mL.

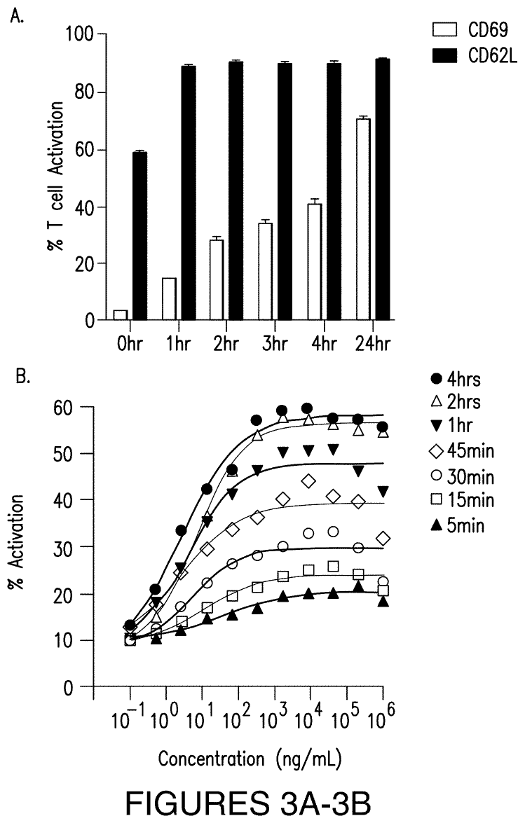

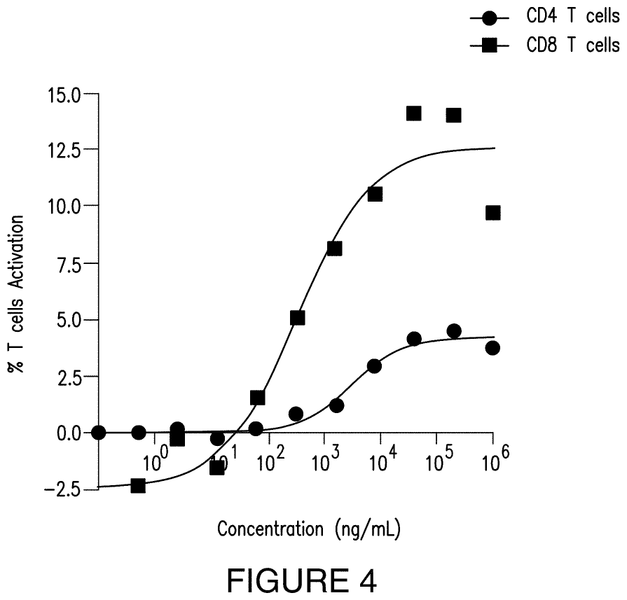

To accurately model initial synapse formation, it can be useful to find the earliest marker of T cell activation. To check for the earliest readout of T cell activation, Jurkat T cells were incubated with BJAB target cells and CD20/CD3 TDB over a period of 24 hours. Cellular activation as detected by CD69 and CD62L expression was measured at various timepoint (FIG. 3A). T cell activation as marked by CD69 expression was detectable one hour, and continued to increase up to 24 hours after addition of CD3/CD20 TDB. In contrast, CD62L shedding reached maximal level of decrease after 1 hour incubation with TDB and target cells (FIG. 3A). CD62L showed a shift in expression as early as 5 minutes after addition of TDB (FIG. 3B). Therefore, CD62L shedding was selected as the early T cell activation marker used to model T cellular synapse formation.

To confirm that the in vitro system utilizing cell lines is an accurate measure of physiological cellular synapse formation between T cells and target cells, PBMCs were isolated from human donors and tested in the synapse assay. CD20/CD3 TDB was added to the PBMCs and incubated for four hours. Both CD4 and CD8 T cells were analyzed for T cell activation using CD62L expression as the marker.

Similar to the activation by Jurkat T cells, CD8 T cells, and to a less extent, CD4 T cell showed TDB dependent activation, indicating that the in vitro assay system with Jurkat T cells is reflective of the in vivo setting with primary human CD4 and CD8 T cells.

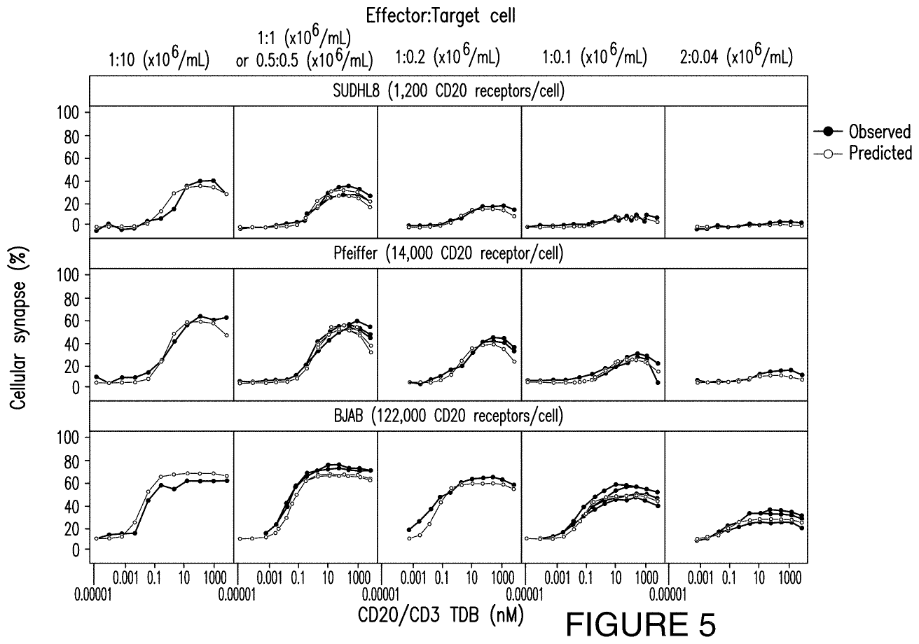

After establishment of a vitro assay system using CD62L expression as the surrogate marker for cellular synapse formation, factors potentially impacting cellular synapse was evaluated. Three B lymphoma cell lines expressing varying amounts of CD20 were used, with BJAB cells expressing the highest level of CD20 (122K copies per cell), Pfeiffer cells expressing intermediate amount of CD20 (14K copies per cell), and SUDHL-8 expressing the lowest amount of CD20 (1.2K copies per cell). In addition, a wide range of effector to target cell ratio (E:T ratio) were evaluated, ranging from (1:10 to 50:1). As shown in FIG. 5, cellular synapse was formed in a target expression dependent manner. BJAB cells, which express the highest levels of CD20, showed the highest amount of cellular synapse, while Pfeiffer and SUDHL8 cells, which express roughly 10- and 100-fold less CD20 respectively, had reduced cellular synapse formation. The target expression level-dependent cellular synapse formation was shown regardless of E:T ratio.

The formation of cellular synapse was also dependent on the relative cell density of effector and target cells (E:T ratio). When cell density of effector cell is 50-fold higher than target cells, minimal cellular synapse formation was observed. By increasing the cell density of target cells, the amount of cellular synapse was elevated (FIG. 5.).

Development of Cellular Synapse Model

A schematic of the proposed cellular synapse model is presented in FIG. 1. The cellular synapse model was developed based on known binding kinetics for TDB, i.e. the formation of tri-molecule synapse complex (i.e. CD20/CD3 TDB-CD2O-CD3) on the surfaces of target and T-cell was required for the formation of cellular synapse, which was approximated by T cell activation markers. The target (i.e. CD20) and CD3 were treated as free and soluble antigens with the binding to TDB as independent event and determined by the binding affinities (i.e. KD). It has been shown that the binding affinity of the mAb could be affected once one of the binding arm was bound, thus an exploratory term, a, was introduced to account for the change of binding affinity. The formation of cellular synapse is then governed by the amount of tri-molecule synapse complex, the distance between target and T-cell, and target expression level per cell.

The ability of the model to characterize and predict the formation of cellular synapse was evaluated using the in vitro T cell activation data with Jurkat T-cell. The cell line was ideal for evaluation of the cellular synapse formation because cell killing function of Jurkat T-cell was impaired even after being activated. Therefore, the amount of the target cell is stationary to allow for quantitation of cellular synapse. A variety of TDB concentrations, effector:target cell (E:T) ratio, and target expression level per cell was included in this dataset to allow for the estimation of model parameters.

As shown in FIG. 5, the model can quantitatively capture the amount of cellular synapse being formed. Based on the mechanism of action for TDB, only the formation of the cellular synapse can trigger the desired downstream activities (e.g. cell killing), while the binding of TDB to either target cell or effector cell alone cannot. Therefore, the model can be used to assist the design of TDB by providing an integrated analysis of the key factors that may affect the formation of cellular synapse

Discussion

T cell dependent bispecific molecules have become a new and promising class of molecules for the cancer treatment. The molecules have a unique MOA, combining tumor target recognition with CD3-mediated T cell recruitment. Enormous efforts have been taken to explore the effects of molecular characteristics and target expression on the anti-tumor activities (e.g. 2:1 target:CD3 binding bispecific molecules, target binding competition with drugs against the same target and used in prior treatment). However, a rational molecular design, target selection, and dose/regimen selection has been challenging due to the lack of quantitative understanding of cellular synapse formation, the driving force of downstream pharmacological effects.

A primary challenge has been measuring the formation of the cellular synapse itself. One approach to model synapse formation would be to image the T cell/tumor cell complexes. However, since the assay is set up in a cell culture dish, the T cells and tumor cells can appear in complex with one another simply due to their close proximity. An alternative approach would be to measure the cell complexes on a flow cytometer, using FSC and SSC measurements to measure the increase in complexes. However, much as with the cell imager, T cell/tumor cell complexes were detected independent of TDB concentration (data not shown).

Rather than measuring the T cell/tumor cell complexes directly, it is possible to measure the cellular synapse formation by measuring the proximal events that follow binding of the TDB to the T cell and tumor cell target. Traditional in vitro assays have measured T cell activation as measured by CD69, CD25, and other cell surface activation markers (Sun, L. L., D. Ellerman, et al. (2015) “Anti-CD20/CD3 T cell-dependent bispecific antibody for the treatment of B cell malignancies.” Science Translation Medicine 7(287): 287ra70; Junttila, T. T., J. Li, et al. (2014) “Antitumor efficacy of a bispecific antibody that targets HER2 and activates T cells.” Cancer Research 74(19): 5561-71; and Brischwein, K., B. Schlereth, et al. (2006) “MT110: A novel bispecific single-chain antibody construct with high efficacy in eradicating established tumors.” Molecular Immunology 43: 1129-1143). Both CD69 and CD62L change in expression on T cells following exposure to TDB in a dose-dependent fashion (FIG. 2). However, CD62L is lost from the surface of T cells within 5 minutes of addition of TDB, and the maximal degree of shedding occurs within 2 hours. This is in contrast to CD69, which continues to increase in expression up to 24 hours after TDB addition (FIG. 3). While both markers show similar sensitivity to TDB (FIG. 2), the changes in CD62L expression are far more proximal to TDB addition and therefore are a more direct readout of cellular synapse formation.

The formation of synapse and the linkage to downstream pharmacological effects have been explored in the previous studies (Brischwein, K., B. Schlereth, et al. (2006) “MT110: A novel bispecific single-chain antibody construct with high efficacy in eradicating established tumors.” Molecular Immunology 43: 1129-1143; Speiss, C., M. Merchant, et al. (2013) “Bispecific antibodies with natural architecture produced by co-culture of bacteria expressing two distinct half-antibodies.” Nature Biotechnology 31: 753-758; and Chen, X., et al. (2016) Mechanistic Projection of First-in-Human Dose for Bispecific Immunomodulatory P-Cadherin LP-DART: An Integrated PK/PD Modeling Approach. Clin Pharmacol Ther. 100(3):232-41). In these studies, formation of synapse was unable to quantitate and was modeled at molecular level with several assumptions: 1) fixed target expression level; 2) fixed CD3 expression level; 3) the calculated total amount of cell-bound target and CD3 are evenly distributed in a well-stirred system as free soluble molecules. The model-predicted molecular synapse was then used to drive the downstream pharmacological effects (e.g. cell killing and T cell dynamics). While this modeling strategy has demonstrated its value in supporting MABEL dose selection and describing the PK-PD relationship in vitro and in vivo, several limitations have been noted.

First, the relationship between the model-predicted molecular synapse and pharmacological effects might be different depending on the target or CD3 expression level per cell. For example, the total amount of the target could be the same under the conditions of i) low cell density of high target-expressing cell vs. ii) high cell density of low target-expressing cell. At certain concentration of bispecific molecules, the amount of model-predicted molecular synapse will be the same, while the pharmacological effects observed could be different due to different cell density. Second, the models used to predict molecular synapse formation assumed the target and the CD3 as free soluble molecules. However, it is anticipated that the bispecific molecule would have different accessibility to cell-bound molecules compared free soluble ones, and thus the cell density needs to be taken into account. Furthermore, given the pharmacologic effects of T cell dependent bispecific molecule were triggered by T cell activation, the relative cell density between target and effector cells needs to be also taken into account. Third, instead of molecular synapse, it is the formation of cellular synapse structure (i.e. bispecific molecules—target cell—T cell) that triggers the downstream pharmacological activities. While formation of molecular synapse (i.e. bispecific molecule—cell-bound target molecule—cell-bound CD3 molecule) on cell surface is prerequisite, the minimal amount of molecular synapse required for cellular synapse structure remains unclear.

The objective of current modeling work is to develop a comprehensive model to describe cellular synapse formation, which was approximated by in vitro assay as described above. The datasets generated cover a wide range of factors potentially impacting cellular synapse formation, including 1) target expression level (1,200 copies per cell ˜122,000 copies per cell); 2) effector to target cell ratio (1:10˜1:0.01); 3) total cell density (1˜11×10⁶/mL). As shown in FIG. 5, the mechanism-based model developed here used a single uniform model structure to describe multiple interrelated factors and their impact on cellular synapse formation. Through the integrated analysis, the model can provide a framework to assist the discovery and development of T cell dependent bispecific molecule, such as molecule design and candidate selection. The information of the dynamic range of tumor target expression level as well as expression difference between tumor and normal cells can also be incorporated to guide suitability assessment of the tumor target and the rational molecule design of the corresponding T cell dependent bispecific molecule. Through the exercise, the therapeutic windows can hopefully be widened by maximizing the tumor cell killing at the site of action and minimizing unwanted immune response and cytotoxicity to normal cells.

### Example 2—Elucidating the MOA of Bispecific Antibodies via a Mechanism-Based Model

This example discloses methodology to measure and predict T cell activation in vitro. It was hypothesized that T cell activation in vitro is a function of: B cell and T cell densities (i.e., intracellular distances), B cell target receptor (CD20) expression levels per cell, and bispecific antibody affinities (KD) for target antigens.

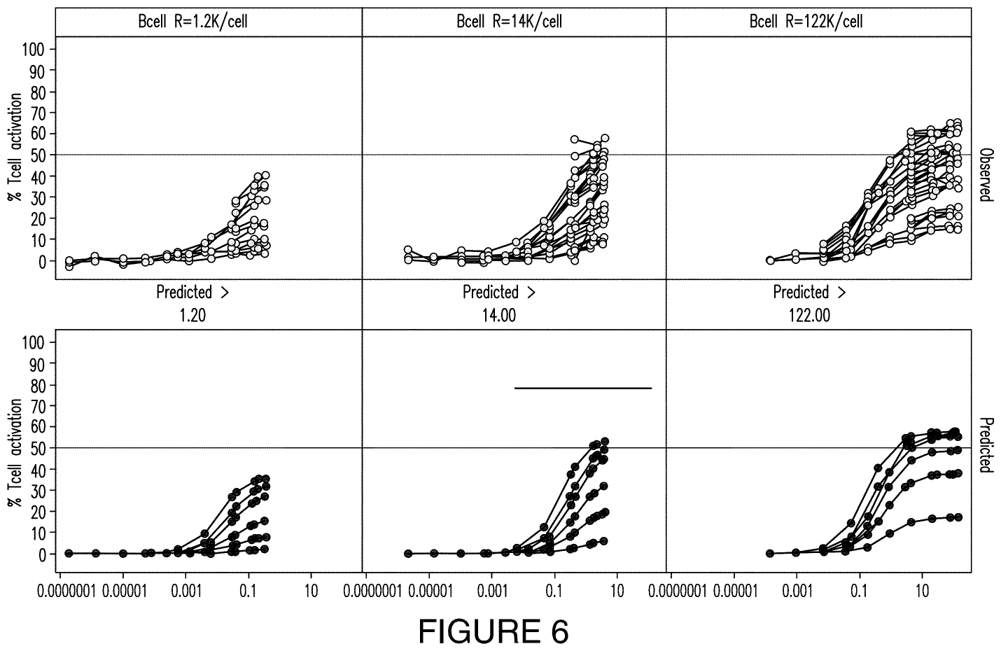

FIG. 6 shows that the T cells are more likely to be activated when B cells had a higher expression level of the antigen CD20.

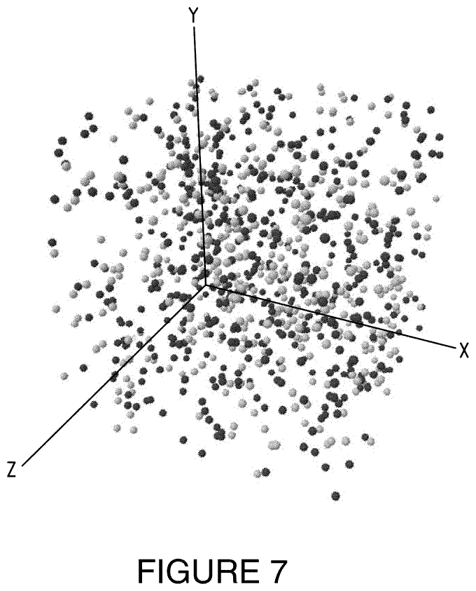

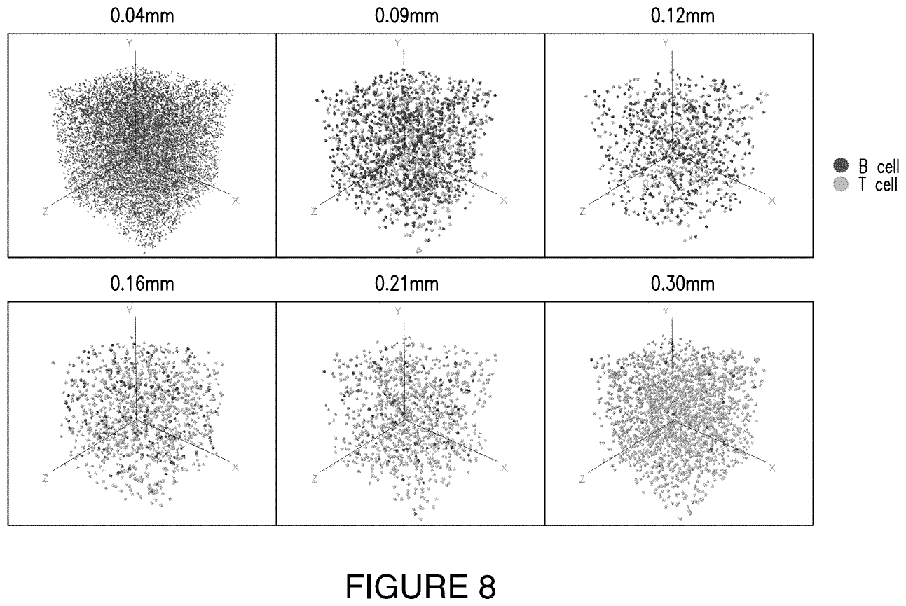

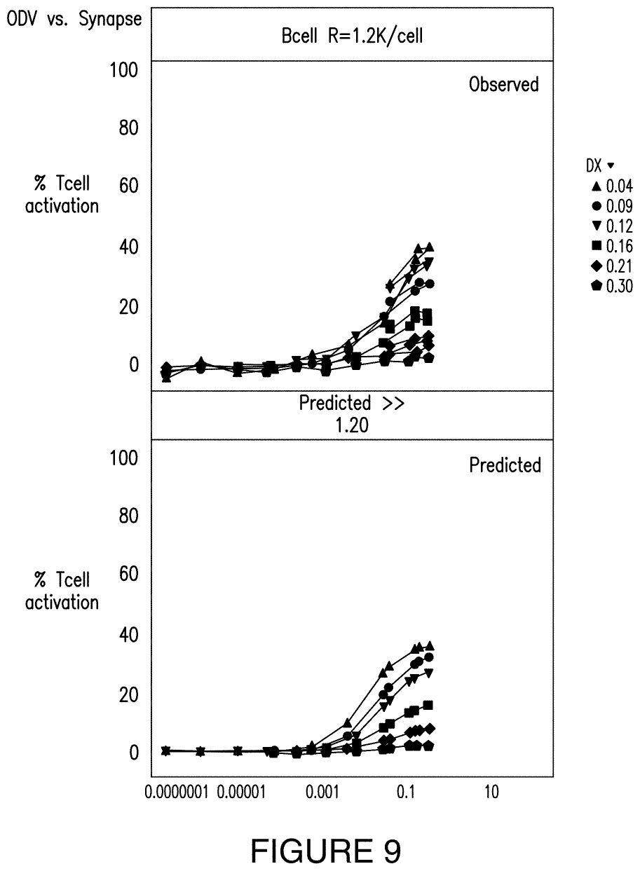

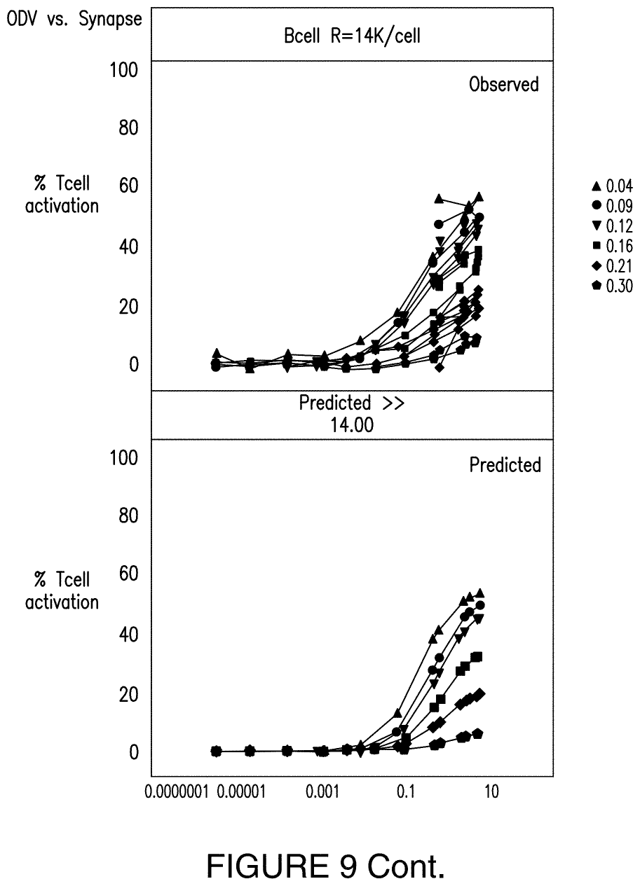

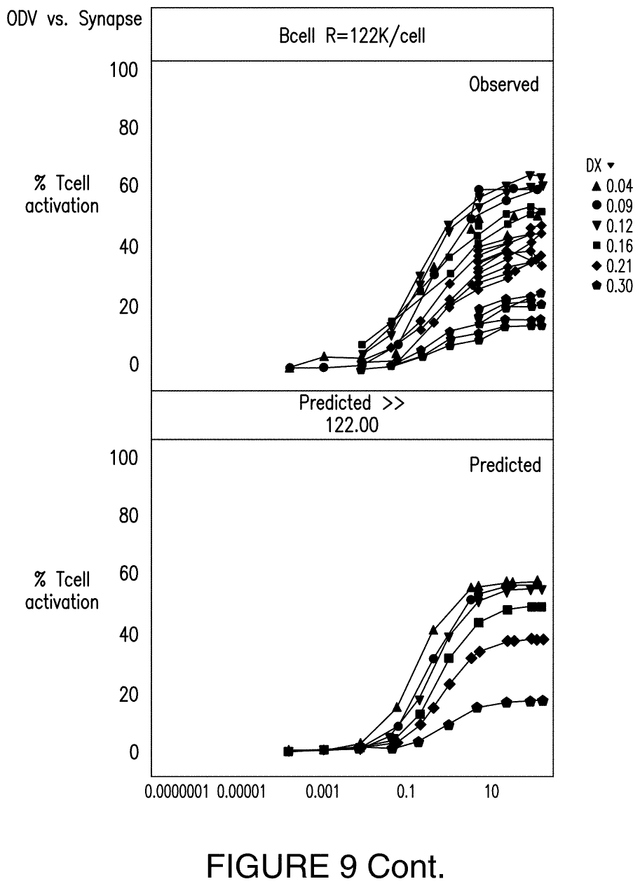

Intracellular distances are useful for modeling as T cells that are closer to B cells are more likely to be “activated” in the presence of a bispecific Ab. Intracellular distance was calculated via simulations. The method to calculate distance between B cells and T cells comprised: using R software to simulate experimental cell numbers with random x,y,z coordinates within a cube having a size of 1 μL (1 mm³); randomly assigning whether a cell was a B or T cell. FIG. 7 shows a simulation of 500 T cells and 500 B cell in 1 μL. For each T Cell (n=500), the average distance (dx) of the 6 closest B Cells was determined. Furthermore, the Overall Average Distance (Dx, in mm) from the previous step was determined to reach a final average distance value. FIG. 8 shows simulations of Intracellular distance between T cells and B cells. FIG. 9 shows that T cells closer to B cells are more likely to be activated.

In summary, higher B cell target expression levels and shorter intracellular distance between T cells and B cells both lead to enhanced T cell activation.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents. Various publications, patents and patent applications are cited herein, the contents of which are hereby incorporated by reference in their entireties

What is claimed is:

1. A method of detecting cellular synapse formation or the activity of a multispecific antibody capable of inducing cellular synapse formation comprising:
(a) contacting a multispecific antibody capable of binding to a first antigen and a second antigen with a first cell expressing the first antigen and a second cell expressing the second antigen, wherein a cellular synapse is formed between the first cell and the second cell upon binding of the multispecific antibody to the first and second antigens; and
(b) measuring activation of the first cell, wherein activation of the first cell indicates cellular synapse formation or that the multispecific antibody is capable of inducing cellular synapse formation.

2. The method of claim 1, wherein measuring activation of the first cell comprising measuring at least one biomarker indicative of activation.

3. The method of claim 1, wherein the at least one biomarker is a cell surface molecule.

4. The method of claim 1, wherein the at least one biomarker is selected from the group consisting of CD62L, CD69, and a combination thereof.

5. The method of claim 1, wherein the first antigen is CD3.

6. The method of claim 1, wherein the second antigen is a tumor antigen selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B.

7. The method of claim 1, wherein measuring activation of the first cell comprises detecting a reporter that is induced upon the activation of the first cell.

8. The method of claim 1, wherein the ratio of the first cell to the second cell is between about 1:10 and about 50:1 or the ratio of the first cell to the second cell is between about 1:10 and about 10:1.

9. The method of claim 1, wherein the average expression of the second antigen on the second cell is at least about 1,000 molecules per cell or at least about 10,000 molecules per cell.

10. The method of claim 1, wherein the average distance between the first cell and the second cell is no more than about 0.3 mm or no more than about 0.1 mm.

11. A system for determining cellular synapse formation of a multispecific antibody that binds to a first antigen and a second antigen, comprising:
(a) a first cell expressing the first antigen;
(b) a second cell expressing the second antigen; and
(c) a means for measuring activation of the first cell, wherein a cellular synapse is formed between the first cell and the second cell upon binding of the multispecific antibody to the first antigen and the second antigen, and wherein the cellular synapse formation activates the first cell.

12. The system of claim 11, wherein the means for measuring activation of the first cell comprises at least one biomarker indicative of activation.

13. The system of claim 11, wherein the at least one biomarker is a cell surface molecule.

14. The system of claim 11, wherein the at least one biomarker is selected from the group consisting of expression of CD62L, CD69, and a combination thereof.

15. The system of claim 11, wherein the first antigen is CD3.

16. The system of claim 11, wherein the second antigen is a tumor antigen selected from the group consisting of HER2, LYPD1, LY6G6D, PMEL17, LY6E, EDAR, GFRA1, MRP4, RET, Steap1, TenB2, CD20, FcRH5, CD19, CD33, CD22, CD79A and CD79B.

17. The system of claim 11, wherein the means for measuring activation of the first cell comprises a reporter gene in the first cell, where expression of the reporter gene is induced upon the activation of the first cell.

18. The system of claim 11, wherein the ratio of the first cell to the second cell is between about 1:10 and about 50:1 or the ratio of the first cell to the second cell is between about 1:10 and about 10.

19. The system of claim 11, wherein the average expression of the second antigen on the second cell is at least about 1,000 molecules per cell or at least about 10,000 molecules per cell or.

20. The system of claim 11, wherein the average distance between the first cell and the second cell is no more than about 0.3 mm or no more than about 0.1 mm.
